(19) United States
(12) Patent Application Publication
(10) Pub. No.: US 2001/0051797 A1
(43) Pub. Date: Dec. 13, 2001

(54) Boudoir hygienic wiping cloth mounting and dispensing apparatus

(76) Inventors: Noel C. Stoute, Hollywood, FL (US); Luisa M. Stoute, Hollywood, FL (US)

Correspondence Address: OLTMAN, FLYNN & KUBLER, 915 MIDDLE RIVER DRIVE, SUITE 415, FORT LAUDERDALE, FL, 33304, US

(21) Appl. No.: 09/803,043

(22) Filed: Mar. 12, 2001

Related U.S. Application Data
(60) Provisional application No. 60/188,666, filed on Mar. 11, 2000.

(51) Int. Cl. A61F 13/15; A61F 13/20

(52) U.S. Cl. 604/385.03; 604/377; 604/385.14; 604/385.15; 604/386; 604/391; 604/392; 604/402

(57) ABSTRACT

A wiping cloth mounting and dispensing apparatus for dispensing a wiping cloth following the sex act includes a wiping cloth; a waist belt sized for wrapping entirely around the waist of a person; a belt securing structure for releasibly securing the belt around the waist of a person; and cloth securing structure removably securing the wiping cloth to the waist belt. The waist belt preferably has a belt middle region and the cloth securing structure preferably includes a hook and loop fastener first cloth patch secured to the waist belt middle region and a corresponding hook and loop fastener second cloth patch secured to the wiping cloth. The wiping cloth preferably is a towel formed of woven cotton towel fabric.

### FILING HISTORY

This application continues from provisional application Ser. No. 60/188,666, filed on Mar. 11, 2000.

### BACKGROUND OF THE INVENTION

1. Field of the Invention

The present invention relates generally to the field of personal hygiene products. More specifically the present invention relates to a wiping cloth mounting and dispensing apparatus including a waist belt and a wiping cloth with cloth securing means for removably securing the wiping cloth to the waist belt for fast and convenient cloth removal and use after sexual intercourse to absorb bodily fluids. The waist belt is preferably an elastic band having corresponding and mating hook and loop belt end fastener patches on opposing sides of the waist ends, so that when the belt is wrapped around the waist of a wearer and the belt ends can be overlapped so that the belt end fastener patches are oriented toward each other, the belt end fastener patches can be engagingly and releasibly pressed together. The cloth securing means preferably includes a hook and loop first cloth fastener patch on the middle region of the waist belt and a corresponding hook and loop second cloth fastener patch on the cloth which releasibly engages the first cloth fastener patch. The wiping cloth is preferably a towel formed of ordinary woven cotton towel fabric. The cloth may be washable and reusable or may be formed of an inexpensive material to be disposable. The preferred embodiment of the apparatus carries only one wiping cloth on the belt at a time.

2. Description of the Prior Art

There have long been dispensers for paper towels, napkins and tissue papers for use in kitchens, restaurant tables, bathrooms and garages. Some of these dispensers are towel and tissue spools rotatably mounted on spool axles extending between and engaging spaced apart wall mounted flanges. Others have taken the form of boxes in which towels or tissues are stacked, having dispensing openings either the box top, side or bottom which permits removal of one towel, tissue or napkin at a time. A problem with these prior dispensers has been that none are well suited to easy reach from a bed during or immediately after sexual intercourse.

It is thus an object of the present invention to provide a wiping cloth dispensing apparatus which can be worn by a person during the sex act and can be easily reached and rapidly removed for absorbing and wiping away bodily fluids released during the sex act for improved personal hygiene and maintaining cleaner sheets.

It is another object of the present invention to provide such an apparatus which is comfortable, has a low profile and is otherwise unobtrusive so that it does not interfere with enjoyment and performance of the sex act.

It is still another object of the present invention to provide such an apparatus which dispenses a wiping cloth with an effortless pull of the cloth, and which permits laundering of the cloth and reattachment for repeated use.

It is finally an object of the present invention to provide such an apparatus which is simple, reliable and highly inexpensive.

### SUMMARY OF THE INVENTION

The present invention accomplishes the above-stated objectives, as well as others, as may be determined by a fair reading and interpretation of the entire specification.

A wiping cloth mounting and dispensing apparatus is provided for dispensing a wiping cloth following the sex act, including a wiping cloth; a waist belt sized for wrapping entirely around the waist of a person; a belt securing structure for releasibly securing the belt around the waist of a person; and cloth securing structure removably securing the wiping cloth to the waist belt.

The waist belt preferably is formed of elastic material. The belt has belt ends and the belt securing structure preferably includes corresponding hook and loop belt end fastener patches secured to opposing sides of the waist belt; so that when the waist belt is wrapped around the waist of a wearer and the belt ends are overlapped so that the belt end patches are oriented toward each other, the belt end patches may be engagingly and releasibly pressed together. The waist belt preferably has a belt middle region and the cloth securing structure preferably includes a hook and loop fastener first cloth patch secured to the waist belt middle region and a corresponding hook and loop fastener second cloth patch secured to the wiping cloth.

The wiping cloth preferably is a towel formed of woven cotton towel fabric, and may be configured and marked to be personalized, to be gender-specific, for designating a special occasion, or to be launderable and reusable. The wiping cloth preferably is formed of inexpensive materials to be disposable. The belt securing structure preferably secures only one towel to the waist belt at a time. The apparatus optionally includes several the wiping cloths.

The wiping cloth and belt are preferably packaged together and sold as a single unit, which is disposable. Piping on the apparatus is preferably provided and is optionally blue or pink, to indicate whether the unit is for men or for women, respectively.

### BRIEF DESCRIPTION OF THE DRAWINGS

Various other objects, advantages, and features of the invention will become apparent to those skilled in the art from the following discussion taken in conjunction with the following drawings, in which:

FIG. 1 is a front view of the apparatus with on waist belt end twisted around to reveal its belt end fastener patch.

FIG. 2 is a bottom, perspective view of the apparatus showing the wiping cloth separated from and positioned for securing to the waist belt, and revealing the first and second cloth fastener patches. The arrows indicate directions of cloth attachment and detachment from the waist belt.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

### First Preferred Embodiment

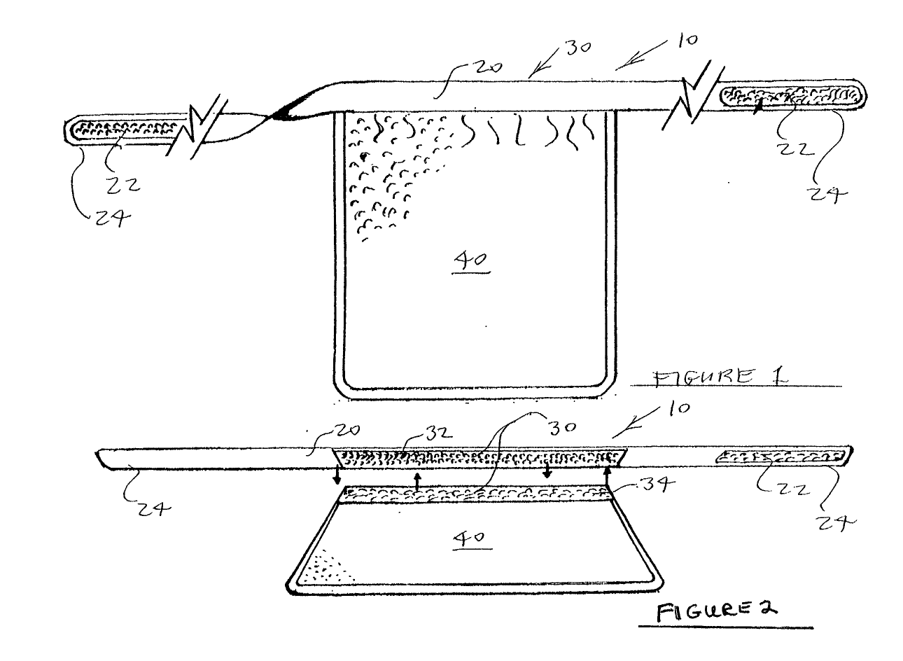

Referring to FIGS. 1 and 2, a wiping cloth mounting and dispensing apparatus 10 is disclosed for cloth removal and use after sexual intercourse to absorb bodily fluids. Apparatus 10 includes a waist belt 20 and a wiping cloth 40 with cloth securing means 30 for removably securing wiping cloth 40 to waist belt 20.

Waist belt 20 is preferably an elastic band such as a “G-string” having corresponding and mating hook and loop belt end fastener patches 22 on opposing sides of waist belt 20 at each belt end 24. As a result, when waist belt 20 is wrapped around the waist of a wearer and belt ends 24 are overlapped so that belt end fastener patches 22 are oriented toward each other, belt end fastener patches 22 can be engagingly and releasibly pressed together. It is contemplated that the belt 20 may be wrapped around a portion of the wearer body other than the waist.

The cloth securing means 30 preferably includes a hook and loop first cloth fastener patch 32 on the middle region of waist belt 20 and a corresponding hook and loop second cloth fastener patch 34 along an edge of the cloth 40. Cloth securing means 30 alternatively can be any suitable fastener which permits quick release, such as snap fasteners (not shown).

Wiping cloth 40 is preferably a towel formed of ordinary woven cotton towel fabric. Wiping cloth 40 may have any of a variety of sizes and shapes and may be configured and marked to be personalized, to be gender-specific or for special occasions. They may be washable and reusable or may be formed of inexpensive materials to be disposable. The preferred embodiment of apparatus 10 carries only one wiping cloth 40 on waist belt 20 at a time, but it is contemplated that several wiping cloths 40 may be secured to the waist belt 20 for sequential removal. Several loose wiping cloths 40 may be provided with apparatus 10.

While the invention has been described, disclosed, illustrated and shown in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

We claim as our invention:

1. A wiping cloth mounting and dispensing apparatus for dispensing a wiping cloth following the sex act, comprising:
a wiping cloth;
a waist belt sized for wrapping entirely around the waist of a person;
belt securing means for releasibly securing said belt around the waist of a person;
and cloth securing means removably securing said wiping cloth to said waist belt.

2. The apparatus of claim 1, wherein said waist belt is formed of elastic material.

3. The apparatus of claim 1, wherein said belt has belt ends and wherein said belt securing means comprises corresponding hook and loop belt end fastener patches secured to opposing sides of said waist belt;
such that when said waist belt is wrapped around the waist of a wearer and said belt ends are overlapped such that said belt end patches are oriented toward each other, said belt end patches may be engagingly and releasibly pressed together.

4. The apparatus of claim 1, wherein said waist belt has a belt middle region and wherein said cloth securing means comprises a hook and loop fastener first cloth patch secured to said waist belt middle region and a corresponding hook and loop fastener second cloth patch secured to said wiping cloth.

5. The apparatus of claim 1, wherein said wiping cloth is a towel formed of woven cotton towel fabric.

6. The apparatus of claim 1, wherein said wiping cloth is configured and marked to be personalized.

7. The apparatus of claim 1, wherein said wiping cloth is configured and marked to be gender-specific.

8. The apparatus of claim 1, wherein said wiping cloth is configured and marked for designating a special occasion.

9. The apparatus of claim 1, wherein said wiping cloth is formed of durable materials to be launderable and reusable.

10. The apparatus of claim 1, wherein said wiping cloth is formed of inexpensive materials to be disposable.

11. The apparatus of claim 1, wherein said belt securing means secures only one towel to said waist belt at a time.

12. A wiping cloth mounting and dispensing apparatus for dispensing a wiping cloth following the sex act, comprising:
a wiping cloth;
a body belt sized for wrapping entirely around a portion of the body of a person;
belt securing means for releasibly securing said belt around the portion of the body of a person;
and cloth securing means removably securing said wiping cloth to said body belt.
